(19) United States
(12) Patent Application Publication
(10) Pub. No.: US 2016/0061923 A1
(43) Pub. Date: Mar. 3, 2016

(54) SHEET TRACTOGRAPHY USING DIFFUSION TENSOR MRI

(71) Applicant: Choukri MEKKAOUI, Boston, MA (US)

(72) Inventor: Choukri MEKKAOUI, Boston, MA (US)

(21) Appl. No.: 14/781,643

(22) PCT Filed: Apr. 3, 2014
(86) PCT No.: PCT/US14/32769

§ 371 Date: Oct. 1, 2015

Related U.S. Application Data
(60) Provisional application No. 61/807,992, filed on Apr. 3, 2013.

(51) Int. Cl. G01R 33/563; A61B 5/00; A61B 5/055; G01R 33/561; G01R 33/56

(52) U.S. Cl. CPC G01R 33/56341; G01R 33/5616; G01R 33/5607; A61B 5/055; A61B 5/0044; A61B 2576/023

(57) ABSTRACT

Systems and methods are provided for performing sheet tractography using diffusion tensor magnetic resonance imaging (“MRI”), Diffusion tensor data acquired from a subject using an MR] system is provided. Eigenvectors and eigenvalues are determined for locations within a subject from the data. Fiber sheets are produced based on these eigenvectors and eigenvalues. For instance, at each location, a fiber sheet is defined to extend along a direction of a first eigenvector, and to have a width that extends along a direction defined by a second eigenvector and a thickness that extends along a direction defined by a third eigenvector. The width and thickness of a fiber sheet can be scaled based on the eigenvalues associated with the second and third eigenvectors, respectively. Metrics, such as a torsion angle that defines the twisting of a fiber sheet, can be determined and mapped based on the fiber sheets.

### CROSS REFERENCE

This application is based on, claims priority to, and incorporates herein by reference in their entirety U.S. Provisional Patent Application Ser. No. 61/807,992 filed Apr. 3, 2013, and entitled “Fiber Sheet Tractography.”

### STATEMENT REGARDING FEDERALLY SPONSORED RESEARCH

This invention was made with government support under HL093038 and RR14075 awarded by the National Institutes of Health. The government has certain rights in the invention.

### BACKGROUND

The present disclosure relates generally to systems and methods for magnetic resonance imaging (“MRI”) and, in particular, to systems and methods for mapping tissue architecture using diffusion-weighted imaging techniques, such as diffusion tensor imaging (“DTI”).

For diffusion MRI techniques, motion sensitizing magnetic field gradients are applied using diffusion weighted imaging (“DWI”) pulse sequences so that the magnetic resonance images include contrast related to the diffusion of water or other fluid molecules. Since microscopic arrangements of tissues often constrain diffusion such that fluid mobility may not be the same in all directions, applying diffusion gradients in several selected directions during the MRI measurement cycle allows diffusion weighted images to be acquired, from which diffusion properties, or coefficients, may be obtained. In the brain, for example, water molecules diffuse more readily along directions of axonal fiber bundles as compared with directions partially or totally orthogonal to the fibers. Hence, the directionality and anisotropy of the apparent diffusion coefficients tend to correlate with the direction of the axonal fibers and fiber bundles. Similarly, in the heart, water diffuses preferentially along myofibers, and so diffusion-encoded imaging techniques allow fiber orientation to be resolved. Hence, application of various processing methods to the diffusion data, allows fibers or fiber bundles to be tracked or segmented, providing indications of normal, injured or diseased tissue construction.

Specifically, in the case of diffusion tensor imaging (“DTI”), three-dimensional distributions of fluid mobility may be represented via tensor field formalism. In order to obtain the apparent diffusion tensor coefficients describing the diffusion tensor, it is generally necessary to acquire at least six DWI images using motion-sensitizing gradients directed in six different directions. Indeed, it may be desirable to acquire more than six directions, but the acquisition of additional DWI images may extend the total scan time. As is known in the art, a diffusion tensor for each voxel provides a reference frame, or eigensystem, that includes orthogonal axes termed eigenvectors, êᵢ, whereby eigenvalues, λᵢ, along the eigenvectors correspond to the degree of diffusivity along each of the major axes of the diffusion tensor. Typically, the orientation of the tensor is commonly taken to be parallel to the principal eigenvector, ê₁, describing the direction of largest diffusion, or the eigenvector associated with the largest eigenvalue, λ₁. For anisotropic fluid diffusion, as observed along tissue fibers or fiber bundles, the principal eigenvector is generally assumed to be collinear with the dominant fiber or fiber bundle orientation.

In particular, heart wall myofibers have been shown to wind as helices around the ventricle chambers, having been resolved by way of histological investigations using sectioned samples, as well as using non-invasive imaging, such as DTI techniques. Presenting additional complication, in vivo data has shown that myofiber architecture is dynamic, as in the case when the left ventricle (“LV”) contracts and relaxes. Microstructural changes in tissues, like the myocardium, are commonly quantified by measuring invariants of the tensors, such as mean diffusivity (“MD”), fractional anisotropy (“FA”), or mode for each location, or voxel, in a region of interest. These invariants provide a basis for comparing tensor components between different tissues or regions. Specifically, the MD describes an average diffusivity, while the FA measures the magnitude of the anisotropic component of the tensor, and the mode describes the type of anisotropy, such as planar anisotropic, orthotropic, or linear anisotropic.

These indices have been widely used in ex vivo cardiac DTI studies of both healthy and diseased myocardium, and have been used in humans in vivo to characterize the microstructural integrity of the myocardium after infarction. Most architecture-related information derived from the DTI data has relied solely upon the diffusion along the principal eigenvector. For example, the helix angle (“HA”) metric relies upon the orientation, or inclination, of the principal eigenvector, while a more recent approach quantifies a propagation angle (“PA”) that measures the angle between two adjacent principal eigenvectors relative to a given myofiber. However, the ability of these metrics to fully characterize structural dynamics during heart activity is limited, and their reproducibility in the human heart in vivo is unknown.

Diffusion MRI tractography is a non-invasive approach that allows for reconstruction of tract trajectories of soft fibrous tissues, such as nerves, muscles, ligaments, and so on, using diffusion tensor data. Traditional methods have involved integrating streamlines defined solely by primary eigenvectors extracted from the diffusion tensors. While such methods supply useful indications regarding fiber assemblies and their respective directionalities, important structural information contained in the rest of the diffusion tensor is ignored. For example, with respect to cardiac tissue, traditional tractography approaches result in cylindrical streamlines that provide no information regarding sheet architecture, which plays a central role in myocardial mechanics.

Therefore, given the above, there is a need for systems and methods directed to improved tissue tractography that fully utilizes the information provided by diffusion tensor imaging or other suitable diffusion-weighted imaging techniques.

### SUMMARY OF THE INVENTION

The present invention overcomes the aforementioned drawbacks by providing systems and methods directed to performing sheet tractography using magnetic resonance imaging (“MRI”) and, in particular, diffusion tensor imaging (“DTI”) or other suitable diffusion-weighted imaging techniques.

It is an aspect of the invention to provide a method for performing sheet tractography using an MRI system. The method includes providing DTI data that has been or is currently acquired from a subject using a MRI system. From the DTI data, a set of eigenvectors is determined for each of a plurality of different locations within a region in the subject. Each set of eigenvectors corresponds to a location in the region and comprises a first eigenvector oriented along a first direction, a second eigenvector oriented along a second direction, and third eigenvector oriented along a third direction. A fiber sheet is produced based on the sets of eigenvectors. For instance, the fiber sheet is defined at each location to extend along the first direction and to have a width extending along the second direction and a thickness extending along the third direction. This process can be repeated to generate multiple fiber sheets in the imaged region. Using the one or more fiber sheets, a tractography map representative of a tissue architecture of a subject can be produced.

The foregoing and other advantages of the invention will appear from the following description.

### BRIEF DESCRIPTION OF THE DRAWINGS

FIG. 1 is a flowchart setting forth steps of an example fiber sheet tractograpy method in accordance with the present invention.

FIG. 2 is an illustration of an example fiber sheet and showing how the fiber sheet can be defined at each location based on three eigenvectors, which may be derived from diffusion tensor data.

FIG. 3 is an illustration showing how a torsion angle of a fiber sheet can be defined.

FIG. 4 is a graphical illustration of myofiber sheet distribution in the lateral left ventricle wall of a human heart.

FIG. 5 is a graphical illustration of heart remodeling resulting from large anteroseptal infarcts.

FIG. 6 is a block diagram illustrating an example of a computer system tgat can implement some embodiments of the present invention.

FIG. 7 is a is a block diagram of an example of a magnetic resonance imaging (“MRI”) system.

### DETAILED DESCRIPTION OF THE INVENTION

By contrast, the present invention affords the ability to capture all information contained in the acquired diffusion tensor data. As will be described, the present invention includes an approach for performing sheet tractography that involves integration of the eigenvectors into ribbon-like, or fiber, sheets, rather than unsealed streamlines. Specifically, a longitudinal orientation, or major axis, at each point along a fiber sheet may be defined based on the direction of a first eigenvector, which may be a primary, or principal, eigenvector of the diffusion tensor. Similarly, transverse orientations of the fiber sheet in directions orthogonal to the major axis may then be defined based on the directions of the second and/or third corresponding eigenvectors which may be secondary and/or tertiary eigenvectors of the diffusion tensor. In addition, transverse dimensions of the fiber sheet may be scaled by the respective eigenvalues of the second and third eigenvectors. In particular, the width of the fiber sheet, extending along a minor axis, may be defined based on the second eigenvalue and its thickness based on the third eigenvalue.

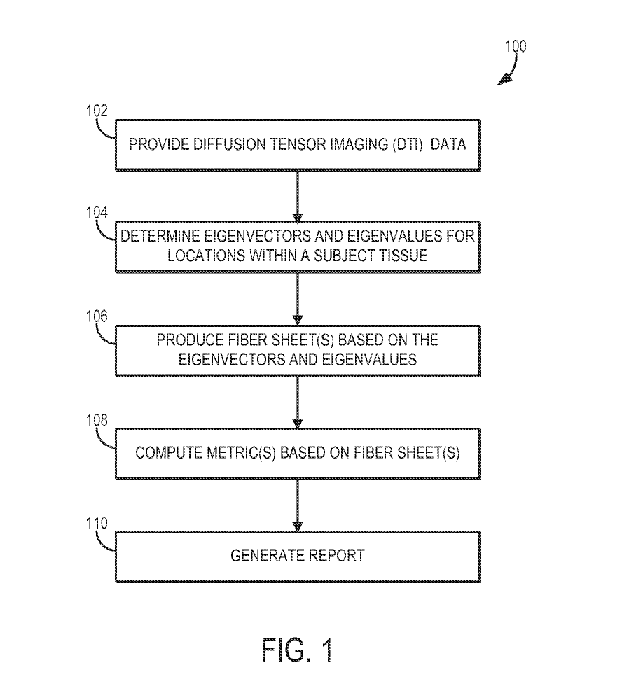

Turning now to FIG. 1, a flowchart setting forth the steps of an example method 100 for fiber sheet tractography is illustrated. The method 100 may begin at process block 102, wherein DTI data, that is or has been acquired from a subject, is provided. As an example, the DTI data may be acquired from a region in the subject that includes a tissue, which may include cardiac tissue. In some aspects, DTI data may include time-series signals representative of, for example, time points or periods of a cardiac cycle, such as diastole or systole periods. In some embodiments, the DTI data may be provided by retrieving the data from a data storage. In some other embodiments, the DTI data can be provided by acquiring it from the subject using an MRI system using any suitable diffusion-weighted pulse sequence. For instance, in one non-limiting example, DTI data may be acquired using a diffusion-weighted, fat-suppressed, single-shot echo-planar imaging (“EPI”) pulse sequence oriented along the short axis of the left ventricle (“LV”), using six different diffusion encoding gradient directions.

Then at process block 104, a set of eigenvectors and eigenvalues may be determined based on the DTI data. For instance, the eigenvectors and eigenvalues can be determined from a diffusion tensor that is computed based on the DTI data. Preferably, a set of eigenvectors and corresponding eigenvalues is determined for each location within the imaged region of the subject, or at least in a tissue in the imaged region. In most instances, each set of eigenvectors includes a first, second, and third eigenvector oriented along respective first, second, and third directions, which are preferably mutually orthogonal.

At process block 106, any number of fiber sheets, sheet tracts, or solid ribbons, may be produced based on the determined eigenvalues and eigenvectors. In general, at each location where a fiber sheet is generated, the fiber sheet extends along the direction defined by the first eigenvector while having a width extending along the direction defined by the second eigenvector and a thickness extending along the direction defined by the third eigenvectors. Preferably, the first eigenvector is a principal eigenvector of a diffusion tensor, the second eigenvector is a secondary eigenvector of the diffusion tensor, and the third eigenvector is a tertiary eigenvector of the diffusion tensor; however, any other suitable permutation or combination of principal, secondary, and tertiary eigenvectors can also be used to define the first, second, and third eigenvectors. In some embodiments, the width of a fiber sheet at a particular location can be determined based on the eigenvalue associated with the second eigenvector at that location. Similarly, in some embodiments, the thickness of a fiber sheet at a particular location can be determined based on the eigenvalue associated with the third eigenvector at that location.

As one example, each fiber sheet may be generated by integrating the first eigenvector extending along a first direction for a number of locations in an imaged region of the subject, and by defining a width and thickness of the fiber sheet based on the second and third eigenvectors. For example, integration of the first eigenvectors to generate fiber sheets can be performed using any suitable numerical integration method, such as a fourth order Runge-Kutta method.

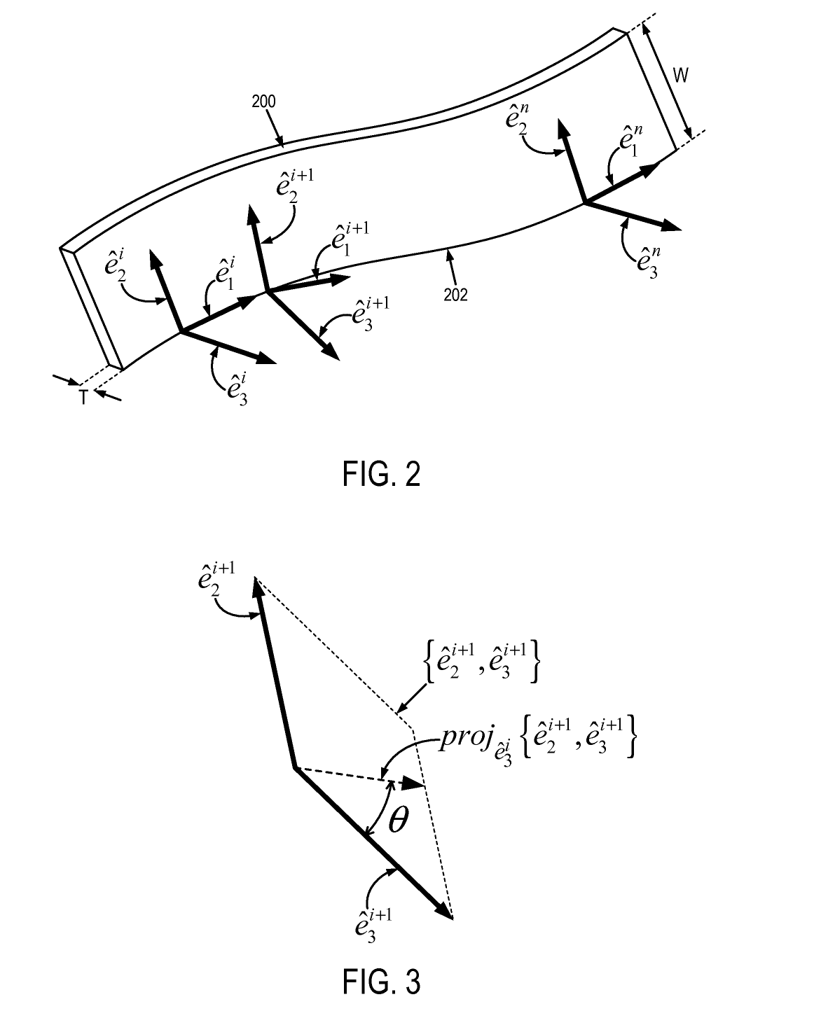

Referring to FIG. 2, an example of a fiber sheet 200 is illustrated. The fiber sheet 200 is generated by first defining a sheet trajectory based on the first eigenvectors, ê₁ⁱ, for i=1, . . . , n, which in this example are principal eigenvectors of the diffusion tensor. In this example, the sheet trajectory coincides with the edge 202 of the fiber sheet 200 on which the origin of the first eigenvectors lie. In some other embodiments, the sheet trajectory 202 could be defined as the centerline of the fiber sheet 200 with the width of the fiber sheet 200 then extending in opposed directions aligned with the second eigenvector.

The width, W, of the sheet is then defined to extend along the direction of the second eigenvector, ê₂ⁱ, at each location along the sheet trajectory. The width, W, of the fiber sheet at each location is determined based on the eigenvalue, λ₂ⁱ, associated with the second eigenvector at that location. For example, the width, W, is scaled based on the eigenvalue. Similarly, the thickness, T, of the sheet is defined to extend along the direction of the third eigenvector, ê₃ⁱ, at each location along the sheet trajectory. The thickness, T, of the fiber sheet at each location is determined based on the eigenvalue, λ₃ⁱ, associated with the third eigenvector at that location. For example, the thickness, T, is scaled based on the eigenvalue.

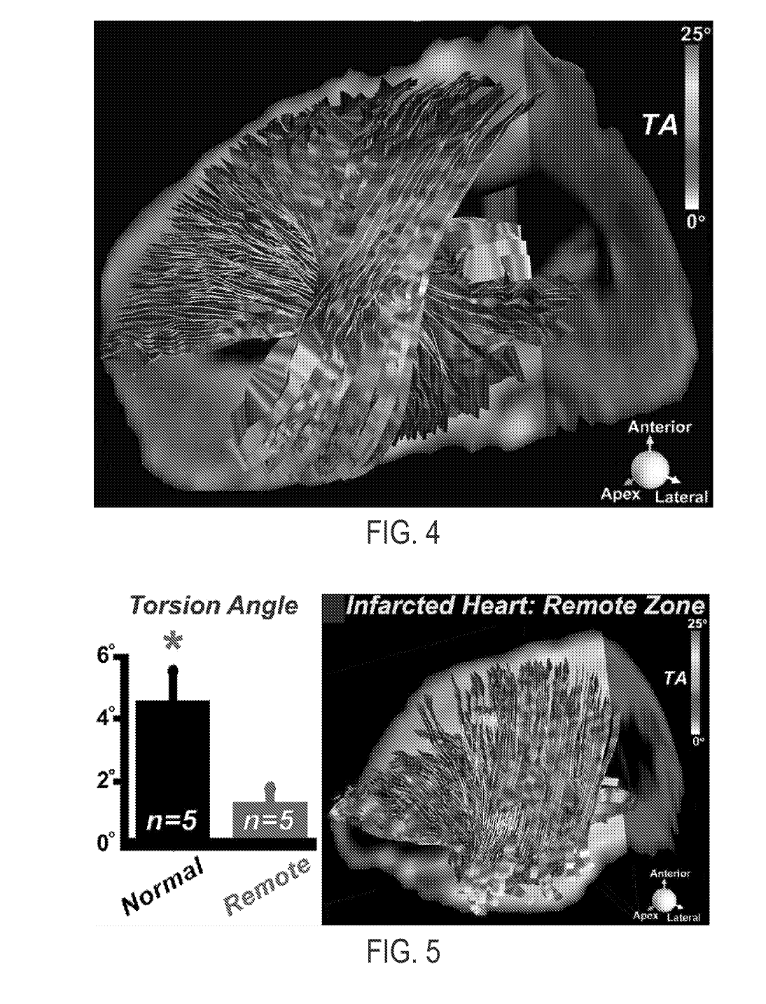

Referring again to FIG. 1, one or more metric can be computed from the one or more fiber sheets, as indicated at step 108. For instance, a torsion angle (“TA”) can be computed at locations along a fiber sheet, or a helical angle or sheet angle can also be computed at locations along a fiber sheet. As shown in FIG. 3, a torsion angle, θ, (or “TA”) can be defined at locations along the fiber sheet. The torsion angle is defined as the angle between vectors normal to the surface of the fiber sheet at two adjacent locations on the fiber sheet. The torsion angle thus represents a degree of local fiber sheet twisting. For example, the torsion angle can be measured between two locations, i and i+1, as the angle between the third eigenvector at the (i+1)^{th} location, ê₃ⁱ⁺¹, and a projection of the third eigenvector at the i^{th} location, ê₃ⁱ, onto the plane, {ê₂ⁱ⁺¹, ê₃ⁱ⁺¹}, defined by the second and third eigenvectors at the (i+1)^{th} location, as illustrated in FIG. 4.

Referring in to FIG. 1, at process block 110 a report may be generated, which could take any desired shape or form. For example, the report may include two or three dimensional maps, tractography maps built using fiber sheets generated as described, and so on. These generated reports thus generally provide indications of tissue fiber architecture and tissue fiber architecture dynamics. In some embodiments, the imaged region of the subject depicts cardiac tissue and, thus, the produced fiber sheets can represent myolaminar sheets describing cardiac tissue. In addition, fiber sheets facilitate derivation of several indices or parameters indicative of fiber sheet architecture for a subject's tissue, as described above with respect to step 108, and hence may be included in reports generated at process block 110. For example, angular differences in orientation between major axes of location points or segments along a fiber sheet may be quantified by a helix angle, while angular differences between the minor axes quantify a sheet angle.

In some aspects, tractography maps included in reports at process block 110 may be color coded, or include a color scale, based on any desired indices, or metrics. For example, FIG. 4 shows the distribution of myofiber sheets in the lateral left ventricle wall of a heart, color-coded by a fiber sheet TA value. The major axis of the fiber sheet follows the helix angle (ê₁) of the myofibers, while the minor axis of the fiber sheet defined by the sheet trajectory (ê₂) is fairly radial.

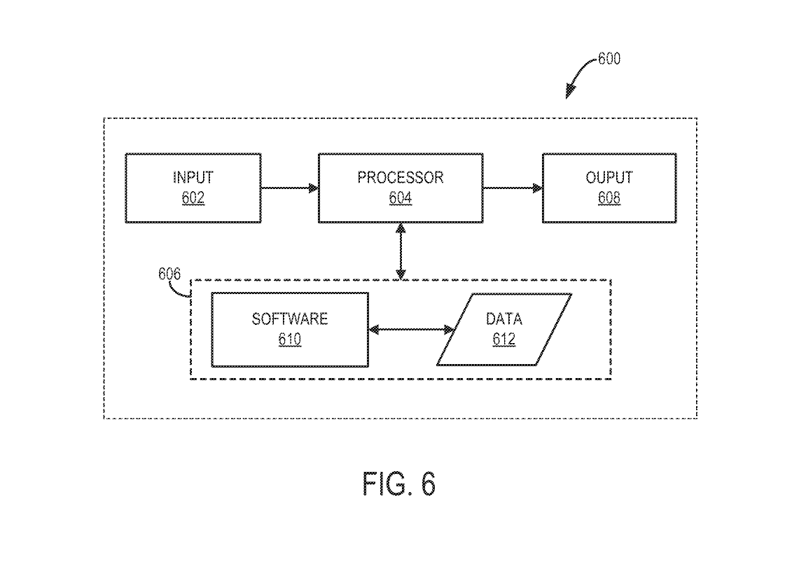

Tractography maps generated using fiber sheets described, may also be advantageously utilized to analyze tissue architecture differences or changes as a result disease or injury. For example, FIG. 6 shows a graphical example comparing normal and remote zone (lateral wall) of a remodeled sheep heart with a large anteroseptal infarct. The average TA is shown to be significantly reduced in the remote zone of the remodeled heart compared to an identical location in a normal heart (p<0.05, Mann-Whitney test). The orientation of the fiber sheets can also be seen to differ between normal (e.g., the heart illustrated in FIG. 4) and infarcted myocardium (e.g., the heart illustrated in FIG. 5. Specifically, the major axes of the fiber sheets have undergone a rightward rotation due to a change in the helix angle (ê₁) in the remote zone. The minor axes of the fiber sheets remain radial but the fiber sheets are now more regular (reduced TA) and closely packed. The reduction in TA in the remote zone of remodeled and/or infarcted hearts may account for the contractile dysfunction in this zone.

Turning now to FIG, 6, a block diagram of an example system 600 that can be used to perform sheet tractography for mapping biological tissue architecture is illustrated. The system 600 generally may include an input 602, at least one processor 604, a memory 606, an output 608, and any device for reading computer-readable media (not shown). The system 600 may be, for example, a workstation, a notebook computer, a personal digital assistant (PDA), a multimedia device, a network server, a mainframe or any other general-purpose or application-specific computing device, or a system in communication with or part of a magnetic resonance system (“MRI”), as will be described. The system 600 may operate autonomously or semi-autonomously, or may read executable software instructions from a computer-readable medium (such as a hard drive, a CD-ROM, flash memory and the like), or may receive instructions from a user, or any another source logically connected to a computer or device, such as another networked computer or server, via the input 602.

The input 602 may take any shape or form, as desired, for operation of the system 600, including the ability for selecting, entering or otherwise specifying parameters consistent with operating the system 600. In some aspects, the input 602 may be designed to accept DTI data acquired from a subject. The input 602 may also be configured to receive other imaging data, such as images that depict regions of the subject's anatomy.

Among the processing tasks for operating the system 600, the at least one processor 604 may also be configured to receive DTI data, wherein the received DTI data may be pre-processed, and/or may undergo any number of further processing steps using the at least one processor 604. In some aspects, the at least one processor 604 may be capable of performing computations using signals derived from DTI data. For example, the at least one processor 604 may be capable of computing from the DTI data, a diffusion tensor at any number of locations, or voxels, within a region of interest. The at least one processor 604 may also be capable of deriving eigenvectors and eigenvalues from such diffusion tensors.

In some embodiments, the at least one processor 604 may be configured to produce any number of fiber sheets by performing computations related to fiber sheet tractography using determined eigenvectors and eigenvalues, as described above. In some embodiments, the at least one processor 604 may be configured to process or perform computations using time-series data. For example, such computations may be representative of, or specific to, time points or periods of a cardiac cycle, such as diastole or systole periods.

In some embodiments, the at least one processor 604 may be configured to compute indices or metrics in relation to the generated fiber sheets. For example, a torsion angle (“TA”) index or metric can be computed at points along a fiber sheet, the torsion angle representing an amount of local fiber sheet twisting and derived from minor axis orientation variations. Specifically, the TA indicates an angle between normal vectors corresponding to adjacent points or segments along each fiber sheet. Other parameters, indices, or metrics, in dependence of determined eigenvectors and eigenvalues, such as the helix angle, sheet angle and no forth, may also be computed by the at least one processor 604.

The at least one processor 604 is preferably configured to create two or three dimensional maps, tractography maps, and so on, indicative of fiber sheet architecture and fiber sheet architecture dynamics for subsequent use, analysis or display via the output 608.

The memory 606 may contain software 610 and data 612, and may be configured for storage and retrieval of processed information and data to be processed by the processor 604. In some aspects, the software 610 may contain instructions directed to producing fiber sheets for any region of interest within a subject's anatomy. The data 612 may include any data necessary for operating the system 600, and may include any raw or processed information in relation to anatomical data, diffusion data, and so forth. In addition, the output 608 may take any shape or form, as desired, and may be configured for displaying, in addition to other desired information, any information in relation to fiber sheet architecture and fiber sheet architecture dynamics. In some aspects, the output 608 may be configured to display two or three dimensional maps, tractography maps, metrics, indices, and so forth, providing indications of fiber sheet architecture and fiber sheet architecture dynamics. For example, tractography maps may be displayed, wherein generated fiber sheet are oriented by their helix angle and/or color coded by corresponding fiber sheet TA values.

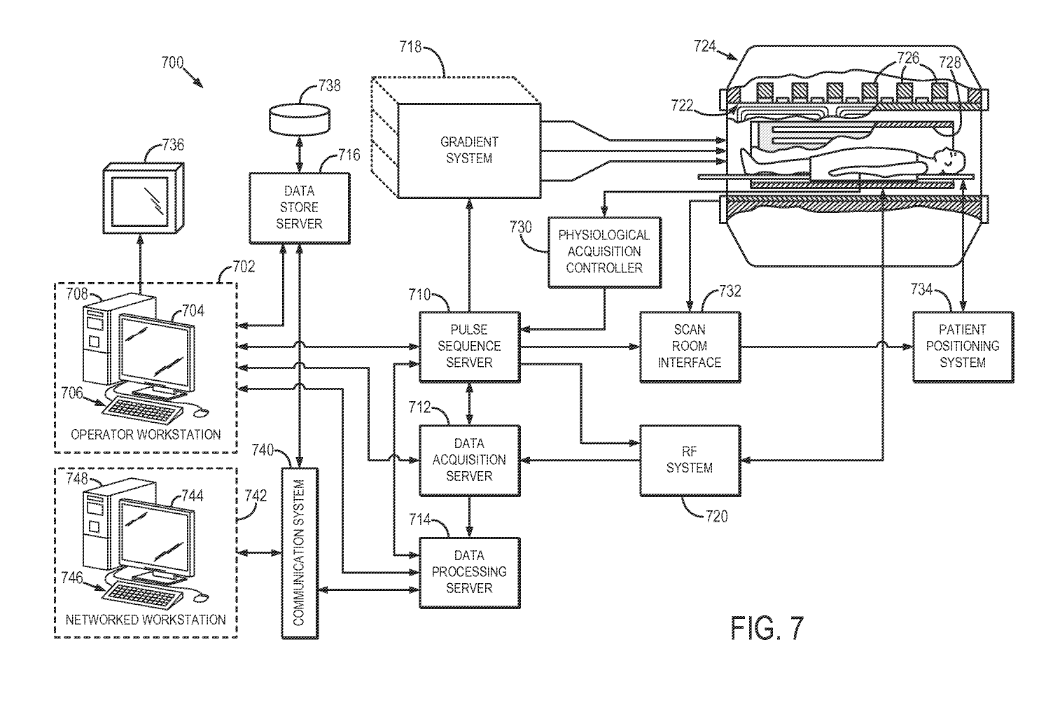

Referring particularly now to FIG. 7, an example of a magnetic resonance imaging (“MRI”) system 700 is illustrated. The MRI system 700 includes an operator workstation 702, which will typically include a display 704; one or more input devices 706, such as a keyboard and mouse; and a processor 708. The processor 708 may include a commercially available programmable machine running a commercially available operating system. The operator workstation 702 provides the operator interface that enables scan prescriptions to be entered into the MRI system 700. In general, the operator workstation 702 may be coupled to four servers: a pulse sequence server 710; a data acquisition server 712; a data processing server 714; and a data store server 716. The operator workstation 702 and each server 710, 712, 714, and 716 are connected to communicate with each other. For example, the servers 710, 712, 714, and 716 may be connected via a communication system 740, which may include any suitable network connection, whether wired, wireless, or a combination of both. As an example, the communication system 740 may include both proprietary or dedicated networks, as well as open networks, such as the internet.

The pulse sequence server 710 functions in response to instructions downloaded from the operator workstation 702 to operate a gradient system 718 and a radiofrequency (“RF”) system 720. Gradient waveforms necessary to perform the prescribed scan are produced and applied to the gradient system 718, which excites gradient coils in an assembly 722 to produce the magnetic field gradients Gₓ, G_{y}, and G_{z} used for position encoding magnetic resonance signals. The gradient coil assembly 722 forms part of a magnet assembly 724 that includes a polarizing magnet 726 and a whole-body RF coil 728.

RF waveforms are applied by the RF system 720 to the RF coil 728, or a separate local coil (not shown in FIG. 7), in order to perform the prescribed magnetic resonance pulse sequence. Responsive magnetic resonance signals detected by the RF coil 728, or a separate local coil (not shown in FIG. 7), are received by the RF system 720, where they are amplified, demodulated, filtered, and digitized under direction of commands produced by the pulse sequence server 710. The RF system 720 includes an RF transmitter for producing a wide variety of RF pulses used in MRI pulse sequences. The RF transmitter is responsive to the scan prescription and direction from the pulse sequence server 710 to produce RF pulses of the desired frequency, phase, and pulse amplitude waveform. The generated RF pulses may be applied to the whole-body RF coil 728 or to one or more local coils or coil arrays (not shown in FIG. 7).

The RF system 720 also includes one or more RF receiver channels. Each RF receiver channel includes an RF preamplifier that amplifies the magnetic resonance signal received by the coil 728 to which it is connected, and a detector that detects and digitizes the I and Q quadrature components of the received magnetic resonance signal. The magnitude of the received magnetic resonance signal may, therefore, be determined at any sampled point by the square root of the sum of the squares of the I and Q components:

M=\sqrt{I²+Q²} (1);

and the phase of the received magnetic resonance signal may also be determined according to the following relationship:

$$ϕ = {tan}^{- 1} ( \frac{Q}{I} ) . \qquad ( 2 )$$

The pulse sequence server 710 also optionally receives patient data from a physiological acquisition controller 730. By way of example, the physiological acquisition controller 730 may receive signals from a number of different sensors connected to the patient, such as electrocardiograph (“ECG”) signals from electrodes, or respiratory signals from a respiratory bellows or other respiratory monitoring device. Such signals are typically used by the pulse sequence server 710 to synchronize, or “gate,” the performance of the scan with the subject's heart beat or respiration.

The pulse sequence server 710 also connects to a scan room interface circuit 732 that receives signals from various sensors associated with the condition of the patient and the magnet system. It is also through the scan room interface circuit 732 that a patient positioning system 734 receives commands to move the patient to desired positions during the scan.

The digitized magnetic resonance signal samples produced by the RF system 720 are received by the data acquisition server 712. The data acquisition server 712 operates in response to instructions downloaded from the operator workstation 702 to receive the real-time magnetic resonance data and provide buffer storage, such that no data is lost by data overrun. In some scans, the data acquisition server 712 does little more than pass the acquired magnetic resonance data to the data processor server 714. However, in scans that require information derived from acquired magnetic resonance data to control the further performance of the scan, the data acquisition server 712 is programmed to produce such information and convey it to the pulse sequence server 710. For example, during prescans, magnetic resonance data is acquired and used to calibrate the pulse sequence performed by the pulse sequence server 710. As another example, navigator signals may be acquired and used to adjust the operating parameters of the RF system 720 or the gradient system 718, or to control the view order in which k-space is sampled. In still another example, the data acquisition server 712 may also be employed to process magnetic resonance signals used to detect the arrival of a contrast agent in a magnetic resonance angiography (“MRA”) scan. By way of example, the data acquisition server 712 acquires magnetic resonance data and processes it in real-time to produce information that is used to control the scan.

The data processing server 714 receives magnetic resonance data from the data acquisition server 712 and processes it in accordance with instructions downloaded from the operator workstation 702. Such processing may, for example, include one or more of the following: reconstructing two-dimensional or three-dimensional images by performing a Fourier transformation of raw k-space data; performing other image reconstruction algorithms, such as iterative or backprojection reconstruction algorithms; applying filters to raw k-space data or to reconstructed images; generating functional magnetic resonance images; calculating motion or flow images; and so on.

Images reconstructed by the data processing server 714 are conveyed back to the operator workstation 702 where they are stored. Real-time images are stored in a data base memory cache (not shown in FIG. 7), from which they may be output to operator display 712 or a display 736 that is located near the magnet assembly 724 for use by attending physicians. Batch mode images or selected real time images are stored in a host database on disc storage 738. When such images have been reconstructed and transferred to storage, the data processing server 714 notifies the data store server 716 on the operator workstation 702. The operator workstation 702 may be used by an operator to archive the images, produce films, or send the images via a network to other facilities.

The MRI system 700 may also include one or more networked workstations 742. By way of example, a networked workstation 742 may include a display 744; one or more input devices 746, such as a keyboard and mouse; and a processor 748. The networked workstation 742 may be located within the same facility as the operator workstation 702, or in a different facility, such as a different healthcare institution or clinic.

The networked workstation 742, whether within the same facility or in a different facility as the operator workstation 702, may gain remote access to the data processing server 714 or data store server 716 via the communication system 740. Accordingly, multiple networked workstations 742 may have access to the data processing server 714 and the data store server 716. In this manner, magnetic resonance data, reconstructed images, or other data may exchanged between the data processing server 714 or the data store server 716 and the networked workstations 742, such that the data or images may be remotely processed by a networked workstation 742. This data may be exchanged in any suitable format, such as in accordance with the transmission control protocol (“TCP”), the Internet protocol (“IP”) or other known or suitable protocols.

Features suitable for such combinations and sub-combinations would be readily apparent to persons skilled in the art upon review of the present application as a whole. The subject matter described herein and in the recited claims intends to cover and embrace all suitable changes in technology.

1. A method for performing tractography using magnetic resonance imaging (“MRI”), the steps of the method comprising:
a) providing diffusion tensor imaging (“DTI”) data acquired from a subject using a MRI system;
b) determining from the DTI data, a set of eigenvectors for each of a plurality of different locations within a region in the subject, each set of eigenvectors corresponding to a location in the region and comprising a first eigenvector oriented along a first direction, a second eigenvector oriented along a second direction, and third eigenvector oriented along a third direction;
c) producing a fiber sheet based on the sets of eigenvectors, the fiber sheet being defined at each location to extend along the first direction and to have a width extending along the second direction and a thickness extending along the third direction; and
d) generating, using the fiber sheet, a tractography map representative of a tissue architecture of a subject.

2. The method of claim 1, wherein step b) includes computing a diffusion tensor at each of the plurality of different locations within the region in the subject and determining the first, second, and third eigenvectors at each location from the diffusion tensor computed at that location.

3. The method of claim 2, wherein the first eigenvector is a principal eigenvector of the diffusion tensor, the second eigenvector is a secondary eigenvector of the diffusion tensor, and the third eigenvector is a tertiary eigenvector of the diffusion tensor.

4. The method of claim 1, wherein step c) includes defining the width of the fiber sheet based on an eigenvalue associated with the second eigenvector.

5. The method of claim 1, wherein step c) includes defining the thickness of the fiber sheet based on an eigenvalue associated with the third eigenvector.

6. The method of claim 1, wherein the tissue is cardiac tissue and the fiber sheet is indicative of a myolaminar sheet in the cardiac tissue.

7. The method of claim 1, wherein step c) is repeated a plurality of times to produce a plurality of different fiber sheets based on the sets of eigenvectors determined in step b).

8. The method of claim 7, wherein the tractography map produced in step d) depicts the plurality of different fiber sheets.

9. The method of claim 1, further comprising computing at least one metric based on the fiber sheet and generating a report that indicates the at least one metric.

10. The method of claim 9, wherein the report indicative of the at least one metric comprises an image that depicts the at least one metric at locations in the subject.

11. The method of claim 10, wherein the at least one metric is at least one of a torsion angle, a helical angle, and a sheet angle.

12. The method of claim 9, wherein the at least one metric is a torsion angle that is computed as an angle between a normal vector on the fiber sheet at a first location and a projected vector at the first location, wherein the projected vector is a projection of a normal vector on the fiber sheet at a second location onto a plane defined by the second eigenvector and the third eigenvector at the first location.

13. The method of claim 12, wherein the report indicative of the at least one metric comprises a torsion angle map that depicts the torsion angle at locations in the subject.

14. The method of claim 13, wherein the torsion angle map includes a color scale defined by the torsion angle.

15. The method of claim 12, wherein the report that indicates the at least one metric comprises color coding locations in the fiber sheet depicted in the tractography map based on the torsion angle.
